(19) United States
(12) Patent Application Publication
(10) Pub. No.: US 2020/0197591 A1
(43) Pub. Date: Jun. 25, 2020

(54) EXTERNAL FUNCTION DEVICE

(71) Applicant: FRESENIUS MEDICAL CARE DEUTSCHLAND GMBH, Bad Homburg (DE)

(72) Inventors: Martin LAUER, St. Wendel (DE); Holger KAMMER, Lebach (DE)

(73) Assignee: FRESENIUS MEDICAL CARE DEUTSCHLAND GMBH, Bad Homburg (DE)

(21) Appl. No.: 16/303,557

(22) PCT Filed: May 5, 2017
(86) PCT No.: PCT/EP2017/060785

§ 371 Date: Nov. 20, 2018

(30) Foreign Application Priority Data
May 20, 2016 (DE) 10 2016 006 089.1

(51) Int. Cl. A61M 1/28; A61M 1/16; A61M 1/34

(52) U.S. Cl. CPC A61M 1/28; A61M 1/16; A61M 2209/08; A61M 2205/121; A61M 1/342

(57) ABSTRACT

The invention relates to an external function device which has at least one housing body having at least one chamber integrated into it for holding medical fluids and at least one locking device for releasably holding an accessory part.

### TECHNICAL FIELD

The present invention relates to an external function device according to claim 1. The invention relates in particular to an external function device comprising at least one housing body with at least one chamber integrated therein for receiving medical fluids and at least one locking device for holding an accessory part releasably.

### BACKGROUND

Cleaning of equipment used in blood treatments can be technically complicated. External function devices such as blood cassettes, for example, are used to ensure adequate hygiene with an acceptable expenditure of labor and also for other reasons.

Such a blood cassette may be designed to fulfill as many functions as possible for preparation and performance of blood treatment methods. Those skilled in the art are familiar with such blood cassettes from WO 2010/121819 or EP 2 227 270, for example. Such blood cassettes are connected to additional accessory parts, for example, valves, medical ports or patient connections or dialyzer connections by means of tubing lines. In practice, the tubing lines are rolled up and secured with strips of adhesive tape, for example, to permit secure packaging and sterilization of the device. When furnishing a dialysis machine with such a blood cassette for a dialysis treatment, these strips of adhesive tape prevent accessory parts from falling on the floor, where they would be contaminated. However, the use of adhesive strips in packaging blood cassettes leads to a greater expenditure of materials and increased labor. When setting up the dialysis machine prior to a treatment, these adhesive strips must be removed and discarded.

### SUMMARY OF THE INVENTION

The object of the present invention is to provide another external function device, which will have simplified handling and therefore increased safety. The external function device may be a blood treatment cassette in particular, but is not limited to these. Other possible embodiments of the present invention include fluid cassettes or pump cassettes for peritoneal dialysis cyclers. In another possible embodiment, the function device is an organizer, a tray or a treatment kit for hemofiltration machines.

The object of the present invention is achieved by an external function device having the features of claim 1.

The external function device has at least one housing body, at least one chamber integrated into the housing body for receiving medical fluids, at least one channel integrated into the housing body to receive and/or carry a medical fluid and at least one valve device entirely or partially integrated into the housing body for controlling or regulating a fluid flowing through the external function device, and at least one locking device for holding an accessory part that is connected to the housing body by a tubing line, so that it is releasable.

The term “housing body” as used herein refers to a three-dimensional body formed from a material suitable for use in a medical treatment method such as a blood treatment method, for example, a plastic material. The housing body can be manufactured by means of a casting method, for example, or an injection-molding method.

The term “chamber” as used herein denotes a volume suitable for receiving at least one medical fluid. The volume may be a closed space or may be surrounded by such a space. However, it may also be an open space or enclosed partially by such an open space and may become a closed space or a space that is closed except for inlet and outlet lines for the fluid only due to the presence of another body - a body other than the housing body.

Chambers may be designed and provided to accommodate valves and/or sensors or the like.

The terms “channel” and/or “line” as used herein denote a device suitable for receiving and/or carrying medical fluids, such as blood, heparin or other medications, saline solution, substituate or the like.

Channels or lines may be designed as structures that are closed or semi-open in some sections. For example, they may be closable by means of a covering device on at least one open side and thus designed so that they can be sealed with respect to components of a blood treatment machine, for example, and/or with respect to the environment. The term “valve device,” as used herein, denotes a device suitable for control or regulation, which can control the flow and/or passage of fluids through channels and/or lines and/or chambers of the external function device. Valve devices can be controlled by means of control or regulating devices provided for that purpose. Triggering may take place automatically, for example. Suitable control or regulating devices may be provided in or on the blood treatment machine, for example.

The phrase “releasable locking,” as used herein, denotes locking means which are designed to be manually releasable connections. They serve to provide a correct spatial arrangement of parts during their manufacture, until further assembly with other parts, for alignment within the complete disposable item and for maintaining the alignment within the packaging of the disposable item from the time of its manufacture up to and including sterilization, storage, shipping and unpacking. They are used to ensure correct handling according to the instructions for use during unpacking the disposable in order to ensure hygiene, for example, as a cough guard and/or as protection against accidental contact and optionally as protection from disinfectants in locations not intended for disinfection and for correct and ergonomically proper furnishing of the treatment machine with the treatment disposable item.

Locking means can prevent disposable components from falling down during the furnishing handling that takes place during equipping, for example. This is true in particular of accessory parts that are connected to a treatment cassette by means of tubing. However, locking means may also be part of the correct mounting of a disposable component during treatment on other disposable components, for example, on a treatment cassette or on locations on the treatment machine provided for this purpose. The existence of the locked holder means that unlockability is also important, in addition to the correct arrangement in space in the locked condition, for example, for the purpose of improved accessibility to an injection site (septum) or for the purpose of detecting the installed state or detecting the switch position of a disposable valve by means of a machine sensor. Unlockability of an accessory part allows the use of the unlocked component at another location at a distance away from the former spatially, for example, for use of a septum and optionally also for the ergonomically and geometrically correct relockability in the old locked positions or possibly in new locked positions that are additionally provided, during the production of the disposable item or during the medical treatment.

However, locking means can also be designed as connections that are not re-releasable. In this case, it may happen that separate production of two parts is less expensive or is necessary from a functional standpoint and the unreleasable locking achieves the actual finished ready-to-use state. Locking may be unreleasable in certain directions of movement but remain releasable in other directions of movement, for example, for rotational movements between the locked parts, and may thus serve to establish a mechanically or sensorially functional system.

The term “accessory part connected to the housing body,” as used herein, refers to an accessory part, such as valves, medical ports or patient connections or dialyzer connections, preferably connected to the housing body by means of a tubing line. For example, a medical port may be an apparatus such as that disclosed in WO 2013/017247. Such a medical port may have a main channel with a lumen for carrying a first fluid through the port, and may also have an opening of a secondary channel for adding a second fluid into the main channel, wherein the port has at least one housing element and at least one operating element that is arranged relative to the housing element, wherein the operating element can be transferred from a first position to a second position, and wherein the port has a sealing section, which is arranged to be rotatable between a first position of the sealing section, in which the sealing section does not close the secondary channel opening or cover it (open position), and a second position of the sealing element, in which the sealing section closes or covers the secondary channel opening in the second position (closed position).

Advantageous refinements of the external function device according to the invention are the subject matter of the dependent claims.

A function device according to the invention may be understood to be a device by means of which functions such as conducting fluid by means of lines, valves, trapping clots and/or the like are possible.

An external function device may be understood to be a device which is not a permanent component of a treatment machine. An external function device is instead connected here to the treatment machine from the outside for the purpose of a treatment.

In a preferred embodiment, the external function device is a blood cassette such as that which those skilled in the art are already familiar from WO 2010/121819.

In a preferred embodiment, the external function device is provided with a covering device, which is part of at least one integrated valve device, on at least one of its surfaces.

In another preferred embodiment, the covering device is connected in at least one section to the housing body in a force-locking and/or form-fitting and/or physically bonded manner. The covering unit may be connected to the housing body, for example, by means of a peripheral weld or some other peripheral connection. Other non-peripheral or spot-type welds or local welds or connections (e.g., adhesive bonding or pressing) of the covering unit to the housing body may also be provided. In other preferred embodiments, the cover device is connected to the external function device in certain regions on both sides of structures (at least one structure). “On both sides” may be understood according to the invention to refer to at least two sides of the respective structure. A connection on both sides may be understood to be at least a double connection in the region, in particular in the immediate region or in the surroundings, in particular the immediate surroundings of the structure.

These structures include, among other things, fluid channels, lines or other elements of the external function device. These preferably include elements, which are designed to be open in a cross section perpendicular to the main plane of extent of the covering unit and/or are covered at least by means of the covering with respect to an exterior or the atmosphere.

The connection on both sides may be welded, for example. It may be fluid-tight, for example, in such a way that no fluid exchange, in particular no exchange of liquid can take place through the region of the connection or joint (region in which the joint has been bonded adhesively or welded). The connection on both sides may be provided for individual fluid channels, lines or other elements, for example, in selected regions of the external function device. It may also be provided for a plurality of such units or for all fluid channels, lines or other elements.

A connection on both sides may refer to a connection on both the left and the right of the corresponding structure. It may thus be provided both at the top and at the bottom, based on the structure or the like.

A connection on both sides may refer to one or two or even more welds along the edge or circumference or the extent of at least one structure or section thereof. A connection on both sides may extend entirely or in sections thereof longitudinally or may be elongated.

In certain embodiments of the present invention, the effort required for pressing the external function device can be reduced advantageously by means of the connection on both sides in certain embodiments of the invention. In many embodiments of the invention in particular, the requirements of precision, with which the external function device is pressed against a blood treatment machine, for example, may be lower in an advantageous manner. In certain embodiments of the invention, a low contact pressure may be sufficient because of the connection on both sides in an advantageous manner. In many embodiments of the invention, a more reliable function of valves, which act on channels covered the connection on both sides, can be achieved advantageously.

The covering unit may be a film in particular.

The film may preferably be a plastic film. In this regard, any laser-weldable film that appears suitable to those skilled in the art is preferably taken into account here.

In another preferred embodiment, the external function device may have connections for connecting it to an extracorporeal circulation so that it will be in fluid communication.

The external function device may be designed in particular as a cassette to and/or for a blood treatment.

The external function device may also preferably be in fluid connection by means of two connectors using at least one peristaltic pump—preferably two—peristaltic pumps. The external function device may have at least one pump tubing segment—preferably two—pump tubing segments and/or may be designed and/or provided for accommodating such a connector.

In another preferred embodiment, the external function device has at least one valve unit, which has at least one web and one segment of the covering unit. The web is designed on the housing body. The web and covering unit are arranged in order to be operable by means of an actuator acting on a web as part of a blood treatment machine for altering the fluid flow in the sense of a valve.

Such a “web” may denote a component, which is integrated into the external function device and/or protrudes away from a surface thereof in any direction. This component may be made of the same material as the external function device. For example, a web may be embodied by means of casting or injection-molding methods during the production of the external function device.

In another preferred embodiment, the fluid flowing through the external function device during its use is a substituate, heparin or some other active pharmacological ingredient, saline solution (in particular 0.9% NaCl solution), blood, air as well as combinations thereof.

The external function device may be connectable to a blood treatment machine in particular. More preferably it may be designed and provided to be connectable to the blood treatment machine by means of a receiving device of the blood treatment machine. The external function device may be coupleable in particular to the surface of the treatment machine facing the covering device.

In another preferred embodiment, the external function device may be coupleable to the blood treatment machine toward the rear at an angle of inclination, preferably between 5° and 11°, in particular at an angle of inclination of essentially or exactly 8°, based on a vertical to the blood treatment machine. The external function device is preferably inclined toward the rear in an upper area thereof (in the use state).

In another preferred embodiment, the external function device has at least one substituate feed site, which has a device for preventing accidental contact and/or a droplet guard. Other sections, in particular any other section of the function device, may also have a droplet guard and/or a device for preventing accidental contact.

A closure function of the ports (one, many or all ports) of the external function device can also be implemented by septums or non-return valves.

The droplet guard may be implemented, for example, by an integrated closing sleeve.

The droplet guard may preferably serve to prevent substituate or blood or a mixture of substituate and blood from running or dripping out of the receiving device of the blood treatment machine during the removal of the external function device. In this way, hygienic handling of the external function device, which has been used and is unclean, can be further ensured, even outside of the treatment machine.

The external function device according to the present invention may be suitable for use in a blood treatment method using a double-needle access or a single-needle access.

A patient's blood is preferably passed through the dialyzer already during the phase when it is being removed from the patient, is dialyzed there, (preferably immediately), after passing through the dialyzer, is stored in the single-needle chamber and returned from there to the patient in the return phase. The blood is dialyzed in the “fresh state,” in which it comes from the patient. It is thus advantageously possible to differentiate the method performed for the blood treatment by means of the cassette according to the invention from any traditional method in which blood is taken from a patient, stored in a separate single-needle chamber, then dialyzed and returned to the patient through a venous air separator.

The external function device may advantageously have at least one single-needle chamber, in which an element for diverting the surge of blood is arranged.

Such a “blood surge-diverting element” or blood surge element may be suitable and provided for achieving a delay in the flow, for creating turbulence and/or for diverting the blood flowing into the single-needle chamber or for eliminating the momentum of the blood surge. Such a blood surge-diverting element may also be designed to be optimized with regard to the flow in particular. For example, it may be designed in the form of an ellipsoidal or round column, which is connected to a wall of the single-needle chamber along at least one portion of its circumference.

Without blood surge-diverting elements, a blood surge flowing through the phantom valve may optionally result in a fountain. This could lead to sloshing movements of the liquid level and/or to the creation of foam. By means of the blood surge-diverting element, the total blood surge may be divided into two smaller blood surges, so that the momentum of the total blood surge can be eliminated and the formation of a fountain, a sloshing movement and/or creation of foam can be advantageously prevented. A blood surge-diverting element may advantageously be used as the blood surge-diverting element, as disclosed in German Patent Application 10 2009 024 466.2 with the title “Receiving Device for Receiving Medical Fluids and an External Function Device and Medical Treatment Machine,” which was filed with the German Patent and Trademark Office by the present applicant on Jun. 10, 2009 for the present invention. Reference is here made to the full content of the disclosure in this regard. The external function device of the present invention may preferably have at least one venous blood chamber.

The single-needle chamber may preferably be arranged above the venous blood chamber, based on the alignment of the external function device during its use.

The venous blood chamber may be divided into at least one upper space and at least one lower space by means of a cross-sectional taper of the housing body.

The upper space and the lower space may be in fluid communication and/or connection with one another.

The upper space may be designed to allow or create a tangential flow of the fluids flowing from the external function device. The upper space may have an area for creating a stable rotational flow of the fluid flowing through the external function device.

The lower space may have a region, which is essentially or completely free of rotational flow of the fluids flowing through the external function device.

Walls or wall sections of the upper space and/or the lower space of the venous blood chamber may preferably be adapted to an inclination of the external function device toward a vertical of the blood treatment machine. This may advantageously be made possible by a fluidically optimized flow or continuous flow of the blood through the venous blood chamber and the ascent of any air that might be present in the blood for the purpose of separation.

The venous blood chamber may be designed and may in particular have an air separation effect as disclosed in the German Patent Application 10 2009 024 465.4 with the title “Air Separation, External Function Device, Blood Circulation And Treatment Machine,” which was filed by the applicant in the patent application with the German Patent and Trademark Office (DPMA) on Jun. 10, 2009. Reference is herewith made to the full content of the disclosure in this regard.

In another preferred embodiment of the external function device, the housing body is designed as a hard part.

The hard part may be a housing body made essentially of one piece and one material. It may be an injection-molded part. It may have a minimum stiffness of more than 400 N/mm², preferably 1200-1800 N/mm² (flexural modulus of elasticity).

In another preferred embodiment of the external function device as a blood treatment cassette, the pressure in the extracorporeal blood circulation can be measured through the covering unit or through the film upstream from the dialyzer.

The external function device may preferably be a disposable item which is discarded after a single use.

### BRIEF DESCRIPTION OF THE DRAWINGS

The present invention is described in greater detail below on the basis of preferred embodiments thereof with reference to the drawings. The same reference numerals in the figures of the drawings denote the same or identical elements.

FIG. 1 shows a side view of an inventive external function device according to a preferred embodiment equipped with a covering unit on its front side;

FIG. 2 shows the external function device from FIG. 1 with the covering unit opened after destructively cutting it open;

FIG. 3 shows the external function device from FIG. 1 and FIG. 2 from the rear side;

FIG. 4 shows schematically the locking device 798 on the function part 1000;

FIG. 5 shows schematically in simplified form the function device 1000 in a view from above with the accessory part 799 locked onto it. The tubing connection between the accessory part 799 and the function device 1000 is not shown;

FIG. 6 shows schematically in simplified form the function device 1000 in a sectional view along the line AB with the accessory part 799 locked onto it. The tubing connection between the accessory part 799 and the function device 1000 is not shown;

FIG. 7 shows in simplified schematic form the accessory part 799 from beneath with the openings 797.

### DETAILED DESCRIPTION OF ONE EXEMPLARY EMBODIMENT

For an exemplary explanation of the present invention, the external function device is a blood cassette, such as that with which those skilled in the art are familiar from WO 2010/121819, for example.

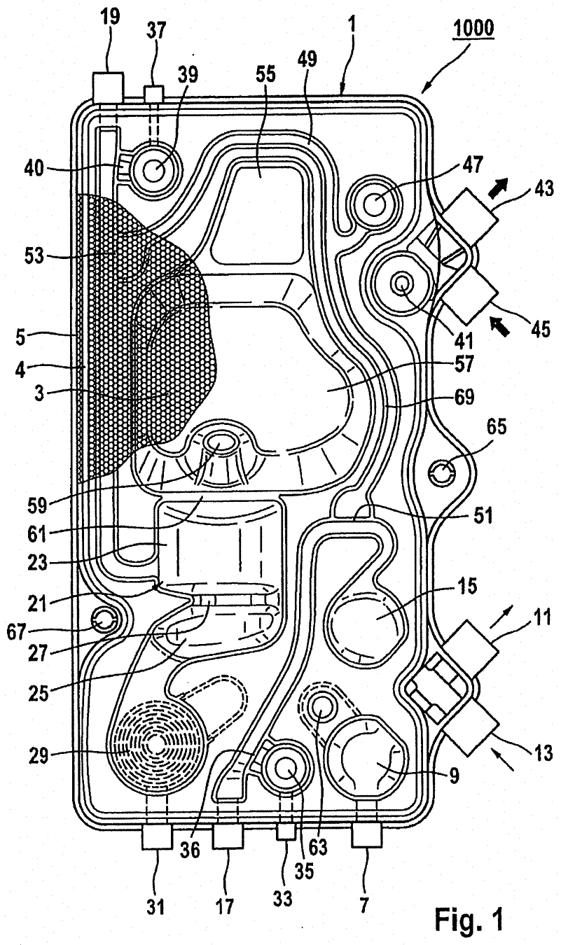

FIG. 1 shows a side view of an external function device, which is provided with a covering unit on the surface, as can be seen in FIG. 1. The external function device is embodied here as a cassette 1000 as an example.

The cassette 1000 has a hard part 1. As shown in FIG. 1 as an example, the hard part 1 has chambers, channels and valves. As additionally shown in FIG. 1 as an example, the chambers, channels and valves are integrated into the hard part 1 and/or embodied at least partially by the hard part 1. The cassette 1000 from FIG. 1 is provided with a covering unit, for example, a film 3 here, on its front side. The covering unit may be welded onto the hard part 1 flatly, i.e., in a planar form.

An embodiment with a three-dimensional design of the welding and sealing contour is also possible according to the invention. The covering unit can seal the chambers and/or channels of the hard part 1 of the cassette 1000, namely with respect to a side of the covering unit facing away from the hard part 1 and/or with respect to the atmosphere.

As shown in FIG. 1, the film is in contact with a peripheral sealing web 4 on the hard part 1 of the cassette 1000. The film 3 is welded to the hard part 1 of the cassette 1000 along a peripheral weld 5.

Alternatively, the peripheral sealing web may be designed to be exposed.

The film 3 may be connected to the hard part 1 of the cassette 1000 at additional local welds (not shown). These may also run peripherally, i.e., so that they are closed in the sense of a sealing border like a ring and/or in spots.

The film 3 may be connected to the hard part of the cassette 1000, e.g., by welding at local spots in a spot weld or a linear weld, in particular at the edge zones of channels carrying liquid.

The film 3 may be connected to the hard part of the cassette 1000 by laser welding. It is advantageous if the local heat input is accomplished by using light-absorbing components. The light-absorbing component may be part of the material of the film and/or of the hard part or a layer which is arranged between the film and hard part or above the film. The layer may be a film layer.

The cassette 1000 may be coupled to a blood treatment machine (not shown in FIG. 1) at least on the front side shown in FIG. 1. An exemplary procedure for suitable coupling of a cassette 1 to a coupling surface of a blood treatment machine is described in German Patent Application 10 2009 012 633.3 with the title “Device for Connecting an External Function Device to an Arrangement, Arrangement Having Such a Device and a Method for Connecting It,” which was filed with the German Patent and Trademark Office on Mar. 10, 2009, and German Patent Application 10 2009 012 632.5, with the title “Sealing Device for Sealing a Volume of a Medical Treatment Arrangement with Respect to Another Volume and the Arrangement and Method,” which was also filed with the German Patent and Trademark Office on Mar. 10, 2009, and reference is herewith made to the full content of these disclosure documents.

The cassette 1000 may be coupled to a coupling surface of the blood treatment machine with the plane of the film 3—or above it. The coupling surface may preferably be designed to be three-dimensional.

The coupling surface of the blood treatment machine may be inclined toward the rear (the direction extending from the observer into the plane of the drawing in FIG. 1) by 8°, for example, relative to a perpendicular line running from top to bottom, for example, on an upper section thereof in FIG. 1.

The cassette 1000 has an arterial patient connection 7.

The cassette 1000 has an arterial pressure measurement chamber 9, which may contain the corresponding sensors. The sensors can preferably transmit signals over a cable line. However, the sensors may also be designed in such a way that they can transmit signals wirelessly.

The cassette 1000 has a connector 11 for the exit of the blood from the cassette 1000 as well as a connector 13 for the admission of the blood into the cassette 1000. The two connectors 11 and 13 can be connected to a pump tubing segment or set of a blood pump.

The cassette 1000 also has a chamber 15 with a pressure measurement site for measuring the pressure in the extracorporeal blood circulation upstream from the dialyzer (“prefilter”) and/or downstream from the pump (“post-pump”).

The pressure in the extracorporeal circulation upstream from the dialyzer can be measured on the chamber 15 through the film 3 and/or above/via the film 3. The cassette 1000 has an arterial filter line 17 and a venous filter line 19.

The interior of the cassette 1000 has a venous blood chamber 21. The venous blood chamber 21 is subdivided into an upper space 23 and a lower space 25.

The upper space 23 of the venous blood chamber 21 can allow a lateral tangential blood inflow. Blood can flow into the upper space 23 through the inlet at the side (the left side in FIG. 1) and can spread tangentially toward the walls of the upper space 23. A lateral tangential blood inflow can create a zone with an essentially or completely stable rotational flow of blood in the upper space 23 of the venous blood chamber 21.

The lower space 25 of the venous blood chamber 21 may form a calming zone for the blood flow. It is possible that there is essentially no rotational flow or no flow at all of blood is present in such a calming zone.

The venous blood chamber 21 is divided by a cross-sectional tapered area 27 of the hard part 1 of the cassette 1000 into the upper space 23 and the lower space 25. The cross-sectional tapered part 27 reduces the cross section of the venous blood chamber 21 in width and depth such that it results in speeding up the flow such that a fluid flowing through the venous blood chamber 21 of the cassette 1000 after crossing through this passage assumes a slower velocity of flow. The upper space 23 and the lower space 25 are in fluid communication. Due to such a construction, i.e., a division of the venous blood chamber 21 into a zone with essentially or completely stable rotational flow of blood and a calming zone for the blood flow, an efficient separation of air from blood or fluid can be achieved advantageously. Walls of the upper space 23 and of the lower space 25 of the venous blood chamber 21 may be adapted in a suitable manner to an inclination of the upper section of the cassette 1000 in FIG. 1 toward the vertical, for example, an inclination of the upper part of the cassette 1000 shown in FIG. 1 by 8° to the rear (into the plane of the drawing). They may be designed to be rounded in a suitable manner so that it advantageously represents a flow-optimized calming surface for fluids which flow through the venous blood chamber 21.

The cassette 1000 has a blood clot catcher 29. Preferably a blood clot catcher such as that disclosed in the invention described in German Patent Application 10 2009 024 495.6 with the title “Blood Clot Catcher, External Function Device, Blood Circulation and a Blood Treatment Machine” by the applicant of the present invention, which was filed with the German Patent and Trademark Office on Jun. 10, 2009, may preferably be used as the blood clot catcher. Reference is herewith made to the full disclosure in this regard.

The pressure in the extracorporeal circulation can be measured on the blood clot catcher 29 through the film 3 and/or above/via the film 3, i.e., in particular after it has passed through the dialyzer.

The cassette 1000 has a venous patient connection 31.

The cassette 1000 has an arterial heparin feed point 33. It should be pointed out here that the heparin feed point 33 (like the venous heparin feed point 37) is also suitable for administering other active pharmacological ingredients in addition to heparin, which are preferably only anticoagulants or may be active ingredient combinations and may be provided for this purpose. This should also always be taken into account when heparin is mentioned in any context above or below.

The cassette 1000 has a non-return valve 35 of the arterial heparin feed point 33. Exemplary non-return valves for use as non-return valve 35 of the arterial heparin feed point 33 as well as the additional non-return valves of the cassette 1000 are disclosed in the Patent Application 10 2009 024 469.7 by the application of the present invention with the title “Valve device, valve insert, external function device, treatment machine and method,” which was filed with the German Patent and Trademark Office on Jun. 10, 2009, to the disclosure content of which in this regard reference is herewith made to the full disclosure content in this regard.

The cassette 1000 has an arterial heparin feed valve 36. With the help of the arterial heparin feed valve 36, the administration of heparin into the arterial filter line 17 can be controlled or regulated.

The supporting webs may serve to support the cassette in the coupled state on a blood treatment machine with respect to a cover of a receiving device of the blood treatment machine for receiving the cassette. Exemplary embodiments of such a coupling of the cassette to the blood treatment machine can be found in German Patent Application 10 2009 012 633.3 with the title “Device for Connecting an External Function Device to an Arrangement, an Arrangement Having Such a Device and a Method for Connecting,” which was filed with the German Patent and Trademark Office on Mar. 10, 2009, the full disclosure of which is herewith referenced in this regard.

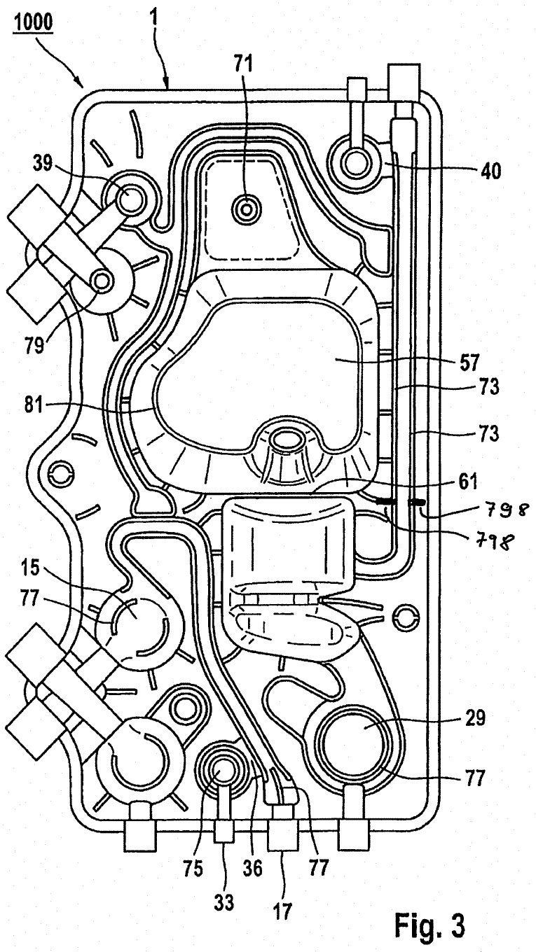

FIG. 3 shows the cassette 1000, as seen by the user/observer looking at it after it has been coupled to the machine interface. The inclination of the cassette 1000 to the machine is designed to be “inclined toward the rear,” so that the upper edge is farther away from the user/observer than the lower edge.

The surfaces of the venous blood chamber 21 facing upward and the single-needle chamber 57 accordingly have an inclination, such that air bubbles can still rise reliably on the inside, despite the inclination of the cassette 1000. Fundamentally of course a cassette design which does not provide any inclination of the cassette is naturally also possible.

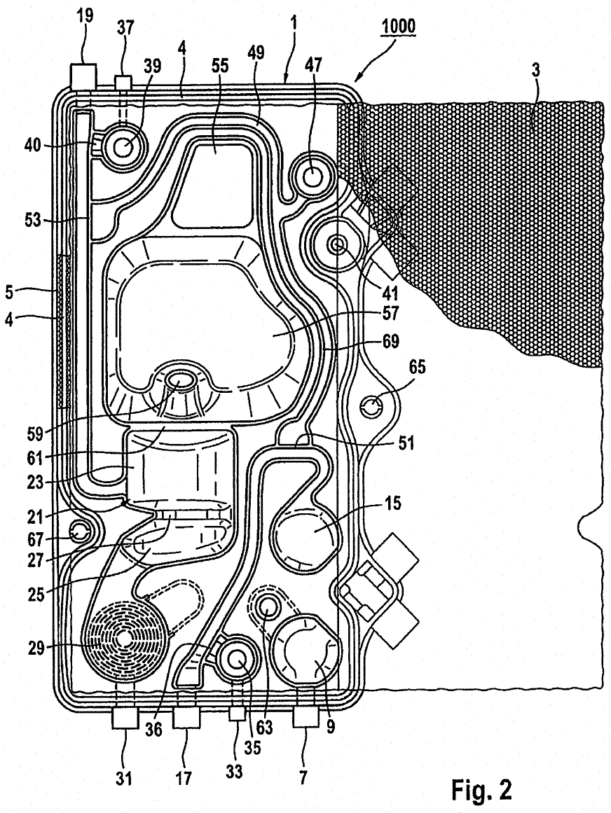

The blood treatment machine has an external function device, for example, the cassette 1000 illustrated in FIGS. 1 to 3 having the elements described above in conjunction with FIGS. 1 through 3.

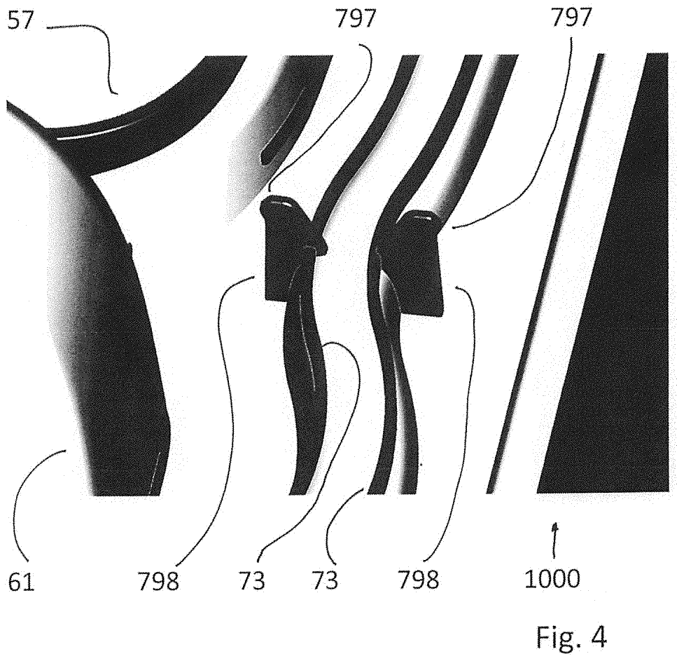

FIG. 4 shows schematically the locking device 798 on the function device 1000. In one embodiment, the locking device 798 has a nose 797 which engages in the openings 796 of the accessory part 799. The accessory part 799 is fastened in a form-fitting and/or force-locking manner on the function device 1000 by means of the noses 797 of the locking device 798.

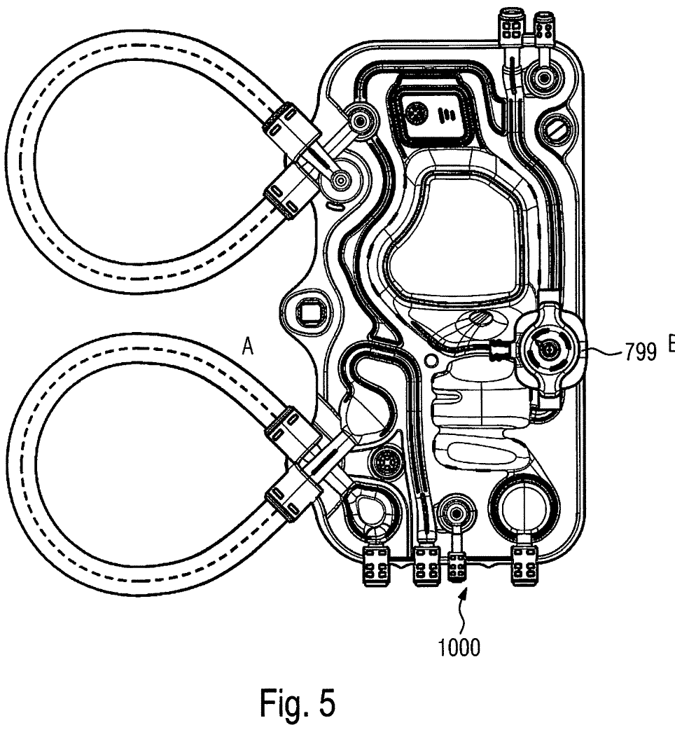

FIG. 5 shows schematically in simplified terms the function device 1000 as seen from above with the accessory part 799 locked onto it. The tubing connection between the accessory part 799 and the function device 1000 is not shown.

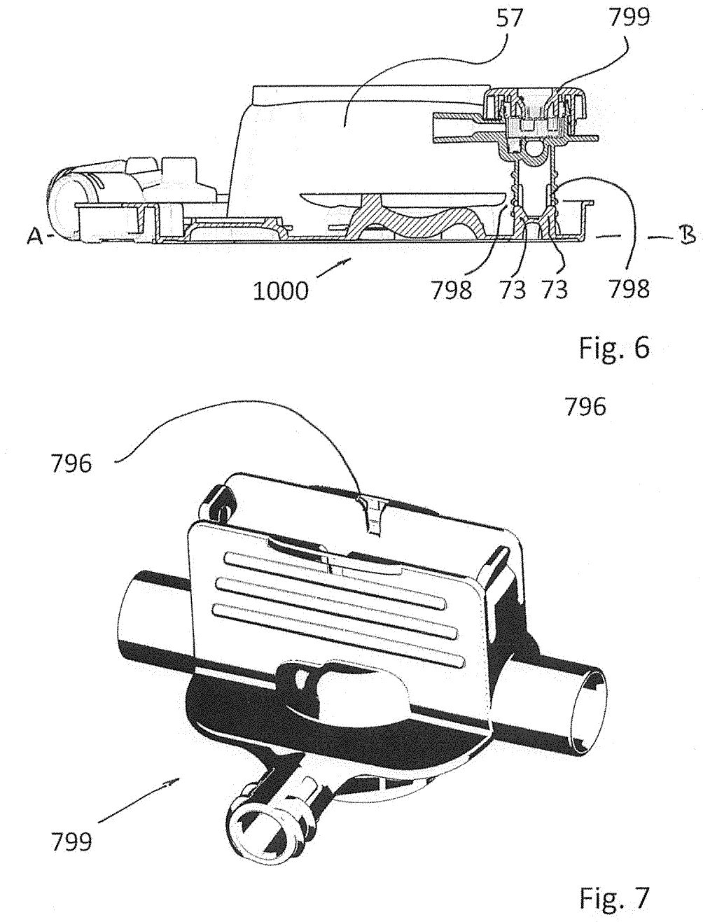

FIG. 6 shows in a schematically simplified form the function device 1000 in a sectional view along the line AB with the accessory part 799 locked onto it. The tubing connection between the accessory part 799 and the function device 1000 is not shown.

FIG. 7 shows in schematically simplified form the accessory part 799 from beneath with the openings 796, which are fastened onto the function device 1000 by means of the noses 797 of the locking device 798 in a form-fitting and/or force-locking manner. The locking of the accessory part 799 on the function device 1000 is preferably designed as a releasable lock.

1. An external function device, comprising:
at least one housing body;
at least one chamber integrated into the housing body for holding medical fluids;
at least one channel integrated into the housing body for holding and/or carrying a medical fluid; and
at least one locking device for releasably holding an accessory part.

2. The external function device according to claim 1, wherein the accessory part is additionally connected to the housing body by means of a tubing.

3. The external function device according to claim 1, wherein the accessory part is a connector, a valve, a hose clamp or an injection point.

4. The external function device according to claim 1, comprising:
at least one housing body, at least one chamber (9, 15, 21, 57) integrated into the housing body for holding medical fluids;
at least one channel (17, 19, 49) integrated into the housing body for holding and/or carrying a medical fluid; and
at least one valve unit (35, 36, 39, 40, 47, 51, 53, 61) which is partially or entirely integrated into the housing body for controlling or regulating a fluid flowing through the external function device, and
at least one locking device (798) for releasably holding an accessory part (799).

5. The external function device according to claim 1, which is provided on at least one of its surfaces with a covering unit, which is part of at least one integrated valve unit (35, 36, 39, 40, 47, 51, 53, 61).

6. The external function device according to claim 1, wherein the cover unit is connected in at least one section to the housing body in a force-locking and/or form-fitting and/or physically bonded manner.

7. The external function device according to claim 1, wherein the cover unit is connected to the housing body by means of at least one peripheral weld (5).

8. The external function device according to claim 6, wherein the cover unit is connected to the housing body by means of additional non-peripheral or point-shaped or local welds.

9. The external function device according to claim 1, which has connections to its connections to an extracorporeal circulation in fluid communication.

10. The external function device according to claim 1, which is designed as a blood treatment cassette (1000), comprising at least one arterial chamber which is integrated into the cassette in a section of an extracorporeal circulation contained therein, and at least one venous chamber, which is integrated into the cassette wherein the cassette (1000) has at least one film (3) as a cover unit and wherein an arterial and/or venous pressure, which prevails in the extracorporeal blood circulation can be measured over the film (3).

11. The external function device according to claim 1, wherein the function device is a blood treatment cassette or a fluid or pump cassette of a peritoneal dialysis cycler.

12. A blood treatment machine, in particular a dialysis machine, characterized in that it is designed to hold at least one external function device according to claim 1.
